(19) United States
(12) Patent Application Publication
(10) Pub. No.: US 2024/0350738 A1
(43) Pub. Date: Oct. 24, 2024

(54) SYRINGES AND INJECTORS WITH CAPACITIVE SENSING LOCKS AND METHODS OF MAKING AND USING SAME

(71) Applicant: Merck Sharp & Dohme LLC, Rahway, NJ (US)

(72) Inventors: John B. Cline, Monroe, NJ (US); Jeffrey C. Givand, Westfield, NJ (US); Guangli Hu, Berkeley Heights, NJ (US); Edward Yeung, Whitestone, NY (US)

(73) Assignee: Merck Sharp & Dohme LLC, Rahway, NJ (US)

(21) Appl. No.: 18/685,627

(22) PCT Filed: Sep. 7, 2022
(86) PCT No.: PCT/US2022/042673

§ 371 Date: Feb. 22, 2024

Related U.S. Application Data
(60) Provisional application No. 63/243,350, filed on Sep. 13, 2021.

(51) Int. Cl. A61M 5/315; A61M 5/20

(52) U.S. Cl. CPC A61M 5/31505; A61M 5/2033; A61M 2005/2073; A61M 2205/13

(57) ABSTRACT

An injecting device comprises a reservoir for containing a medicament, a needle in communication with the reservoir and configured to deliver the medicament to a patient's body, a capacitance sensor disposed adjacent to the needle, and a plunger rod assembly comprising a housing and a piston, the housing and piston being configured to transition between a locked condition in which the piston and the housing are coupled to move together, and an unlocked condition in which the piston is capable of translating relative to the housing.

### FIELD OF THE INVENTION

The present disclosure relates generally to syringes and injectors having capacitive sensing capabilities. More specifically, the present disclosure relates to syringes and injectors having capacitive sensing used for assessing insertion of a needle into a patient's body.

### BACKGROUND OF THE INVENTION

A pre-filled syringe typically includes a glass barrel containing a pharmaceutical product, which is sealed by a stopper. One concern when using pre-filled syringes is known as “dose splitting,” in which the contents of, typically, a prefilled syringe designed for subcutaneous injection is transferred into another container, such as a vial or intravenous bag, in preparation for off-label or otherwise unintended use. Behavior such as “dose splitting” is undesirable, and may undermine data collection, for example, for clinical trials. Additionally, conventional devices and methods do not provide sterile and accurate techniques for assessing patient compliance.

Thus, there exists a need for devices that improve upon and advance the methods of safely using injectors and syringes, such as pre-filled syringes.

### SUMMARY OF THE INVENTION

In one embodiment, an injecting device includes a reservoir for containing a medicament, a needle in communication with the reservoir and configured to deliver the medicament to a patient's body, a capacitance sensor disposed adjacent to the needle, and a plunger rod assembly including a housing, and a piston; the housing and piston being configured to transition between a locked condition in which the piston and the housing are coupled to move together, and an unlocked condition in which the piston is capable of translating relative to the housing.

### BRIEF DESCRIPTION OF THE DRAWINGS

Various embodiments of the presently disclosed syringes and sensors are disclosed herein with reference to the drawings, wherein:

FIG. 1 is a schematic front view of a pre-filled syringe having a capacitive sensor;

FIG. 2 is a schematic perspective view of a pre-filled syringe and a plunger rod assembly;

FIG. 3 is a schematic exploded perspective view showing the components of one example of a plunger rod assembly; and

FIGS. 4-7D illustrate another embodiment of a plunger rod assembly and some variations of latch fingers.

Various embodiments are described below with reference to the appended drawings. It is to be appreciated that these drawings depict only some embodiments of the disclosure and are therefore not to be considered limiting of its scope.

### DETAILED DESCRIPTION OF THE INVENTION

Despite the various improvements that have been made to injectors and syringes, such as pre-filled syringes, conventional methods suffer from some shortcomings as discussed above.

Therefore, there is a need for further improvements to the devices and methods used to deliver medication and measure patient compliance. Among other advantages, the present disclosure may address one or more of these needs.

As used herein, the term “proximal,” when used in connection with a component of a syringe or injector, refers to the end of the component closest to the user's hands when holding the device; whereas the term “distal,” when used in connection with a component of a syringe or injector, refers to the end of the component closest to the needle insertion site during use.

Likewise, the terms “trailing” and “leading” are to be taken as relative to the operator's fingers (e.g., physician) of the syringe or injector. “Trailing” is to be understood as relatively close to the operator's fingers, and “leading” is to be understood as relatively farther away from the operator's fingers.

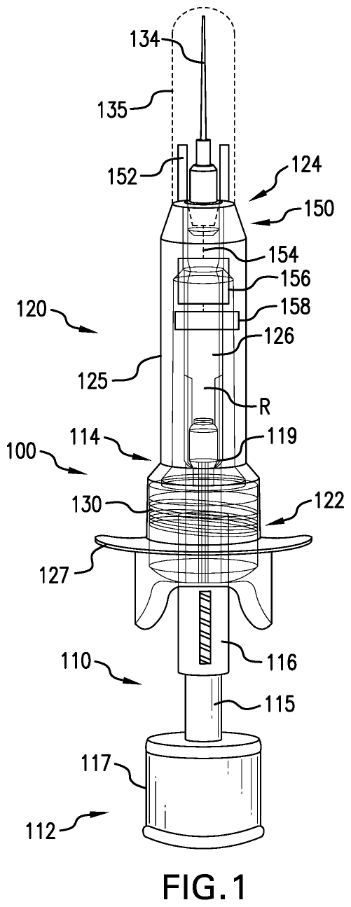

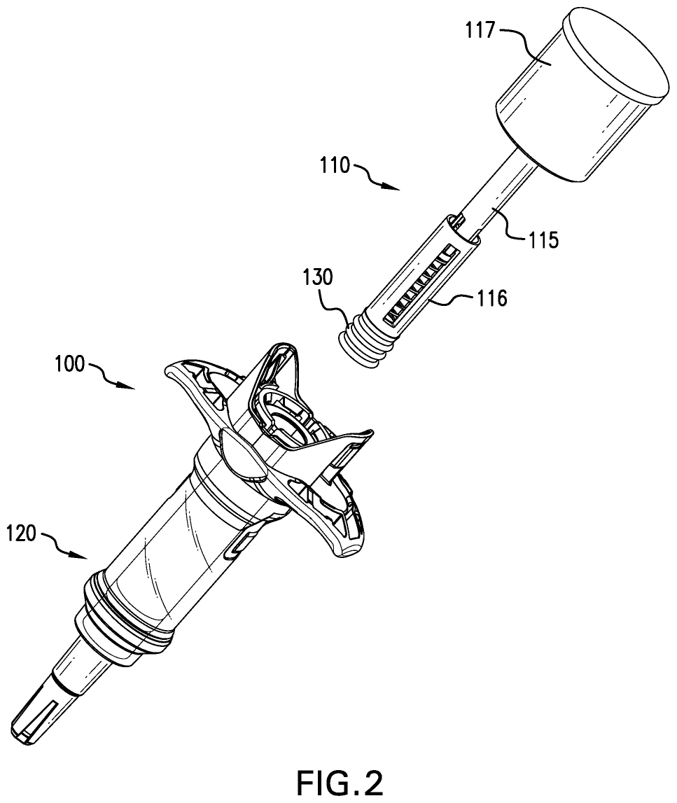

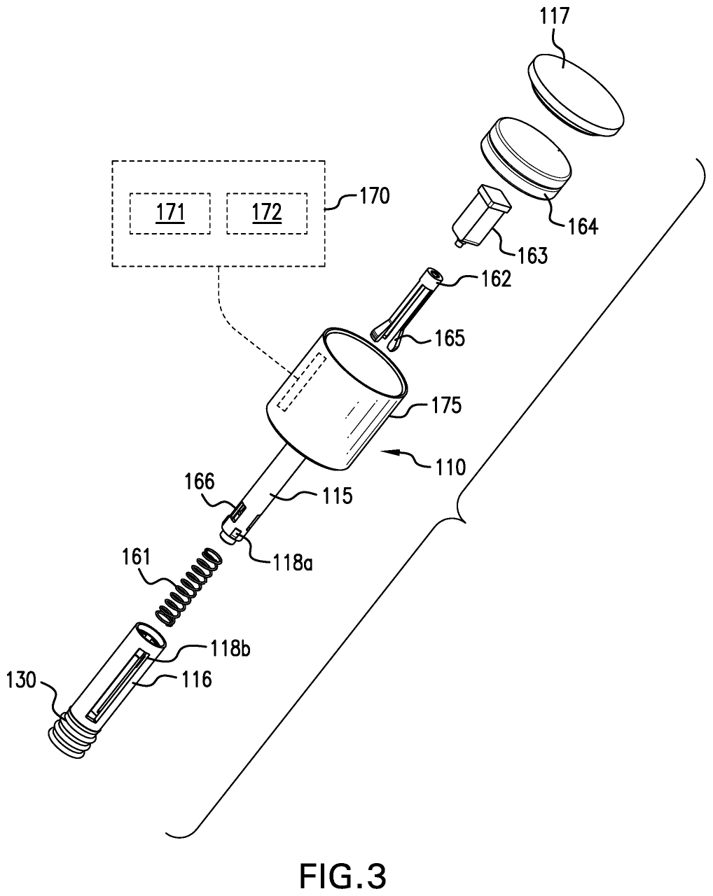

Reference is now made to FIG. 1, which shows an exemplary prefilled-syringe 100 contained within a needle safety device. It will be understood that though a needle within a safety device is shown, the disclosure is not thus limited. For example, though a pre-filled syringe with a staked needle is shown, it will be understood that the principles disclosed herein are equally applicable to other types of injectors (e.g., syringes with removable needles, auto-injectors, or on-body (wearable) injectors, etc.). Pre-filled syringe 100 generally comprises two main portions, a plunger rod assembly 110 and a barrel 120. Plunger rod assembly 110 generally extends between a proximal end 112 and a distal end 114, and generally comprises an elongated piston 115 and a housing 116 extending between a plunger flange 117 and a stopper 130. Additional details of plunger rod assembly 110 will be described with reference to FIGS. 2-3.

A cylindrical barrel 120 extends between proximal end 122 and distal end 124 and comprises a body 125 defining a lumen 126 for accepting a portion of plunger rod assembly 110. Body 125 further comprises a barrel flange 127 adjacent proximal end 122 and defines a reservoir “R” that holds a medicament, drug, saline, or other substance for injecting into a patient's body. An internally threaded stopper 130 is disposed inside lumen 126 of body 125. In one embodiment, stopper 130 is made of an elastomeric material such as natural rubber, synthetic rubber, thermoplastic elastomers, or combinations thereof, and comprises an opening to receive and mate with a portion of plunger rod assembly 110 by advancing a portion of the plunger rod assembly inside the barrel lumen 126 and rotating at least one of coupler 119 and stopper 130 relative to the other.

In this example, a cap 135 is disposed over needle 134. Once cap 135 is removed, the user may pierce the patient's skin with the needle, then push on plunger flange 117 to drive the plunger to deliver a medicament through needle 134 into the patient's body.

Syringe 100 of FIG. 1 further illustrates the use of a capacitive sensor system to increase the safety of the device. Specifically, sensing system 150 may help to electrically detect that the needle of a syringe has been injected into human or animal tissue. In some examples, the detection system and corresponding method use a capacitance sensing approach in which the syringe's needle (or cannula) is indirectly capacitively coupled (not in conductive contact) to one or more sensor electrodes and signal processing circuitry. A pair of electrodes made of a metal, such as copper, titanium, brass, silver, and platinum, or other suitable metals, alloys, conductive inks or polymers or materials, may be used. Electrodes 152 are shown as being two relatively flat plates, but it will be understood that the shape and/or size of the electrodes may be varied as desired. Electrodes 152 may be in electrical communication via wires 154 to a capacitance-to-digital converter (CDC) circuit 156 or similar suitable systems that employ a variety of detection methods including charge transfer (e.g., analog measuring techniques, analog signal converted to digital signal through the use of convention analog-to-digital converters, integrated circuits, etc.). Circuit 156, in turn, may be electrically coupled to a power source (not shown) and a microcontroller 158 having a processor, storage capability and/or wireless transmitting capability, for example, to store data or relay it to a computer or other suitable system.

Turning to FIG. 2, syringe 100 has been unassembled to show the barrel 120 and plunger rod assembly 110, which is configured to be at least partially received within the barrel and made to translate relative thereto. Generally, telescoping plunger rod assembly 110 is capable of transitioning between a locked state or condition and an unlocked state or condition. This transition between the two states may be based, at least partially, upon the detection of tissue using the sensors previously described. Elongated piston 115, housing 116, plunger flange 117 and stopper 130 are all shown in FIG. 2, but additional components of plunger rod assembly 110 are illustrated in the exploded view of FIG. 3.

Specifically, housing 116 may allow for the elongated piston 115 to have a telescoping action along at least a portion of the length of housing 116. Spring 161 is disposed within housing 116 and may be compressed by piston 115 as it advances within housing 116. Two mating features 118a-b are disposed on piston 115 and housing 116, respectively, and configured to retain or otherwise couple the two components together when spring 161 is in its uncompressed or fully extended condition. When needle 134 (not shown) touches or enters tissue, a wireless signal is sent from a capacitive sensor in, or adjacent, the cannula of the syringe to a receiver (not shown) in the plunger rod assembly. The received signal, in turn, activates a miniature solenoid 163, the solenoid 163 being configured to axially drive latch 162 comprising one or more fingers 165. A latch having three fingers is shown, although it will be understood that a single finger may be used as well as latches having two, three, four, five or more fingers.

In one example, the latch fingers 165 are longitudinally driven down the inner diameter of piston 115 until the fingers 165 protrude outwards from the openings 166 and engage onto the rigid features on housing 116. In one embodiment, the number of openings 166 may be the same as the number of fingers on the latch. Once the fingers 165 are engaged in openings 166, they are held in place and cannot be retracted. The fingers 165 also engage features on the outer plunger housing 116, inhibiting the piston 115 from moving longitudinally within housing 116. With the two components (i.e., piston 115 and housing 116) being fixed and/or immovable relative to one another, the plunger rod assembly 110 is in the “locked” condition that prevent translation or telescoping of one component relative to the other. In this locked condition, the plunger rod assembly 110 is rigid and allows the user to deliver a partial or full dose of the pre-filled syringe. Alternatively, in the unlocked condition, the piston 115 and housing 116 can translate relative to one another and stopper 130 does not eject the contents of the barrel via needle 134.

Miniature solenoid 163 may be disposed inside piston 115 along with one or more batteries 164 electrically in communication with the solenoid to power it and actuate the latch. Also contained in the plunger rod 115 are electronics 170, which may include a receiver circuit 171 to receive a signal from the capacitive sensor that is generated when the sensor detects contact or entry of the needle into the patient's tissue. In one embodiment, a printed circuit board 172 may also be used to control the solenoid. In one embodiment, solenoid 163, batteries 164, and circuit board 172 may be housed within an enlarged casing 175 of piston 115. In one embodiment, the enlarged cylindrical casing 175 of piston 115 houses solenoid 163, batteries 164, and circuit board 172 which are held in place by locating features in the plunger rod housing 115, and plunger flange 117 which serves as a cap. Plunger flange 117 may be snapped into place, pressed into place, or permanently bonded by welding or adhesive means to enlarged casing 175. When entry of the needle into tissue is detected, the solenoid 163 is energized, the latch is actuated, and piston 115 and housing 116 are linked together to enable dispensing of the contents of the syringe.

In one embodiment, to ensure proper function of the mechanism described above and safety with respect to sterility, housing 116, piston 115 and flange 117 may comprise plastics, which may include acrylonitrile butadiene styrene (ABS), polypropylene, polycarbonate, and/or polyethylene. Spring 161 may be made from stainless steel or other suitable material. Latch 162 including fingers 165 may comprise an engineered plastic such as ABS or polycarbonate. Such plastics may comprise fillers to enhance their strength under load. Such fillers may include, but are not be limited to, glass beads or glass fibers. In one embodiment, the latch fingers may comprise a metal, such as stainless steel. In one embodiment, the latch fingers may comprise both plastic and metal, created by insert molding or by a secondary operation. Such a secondary operation could consist of bonding or heat staking plastic and metal elements together.

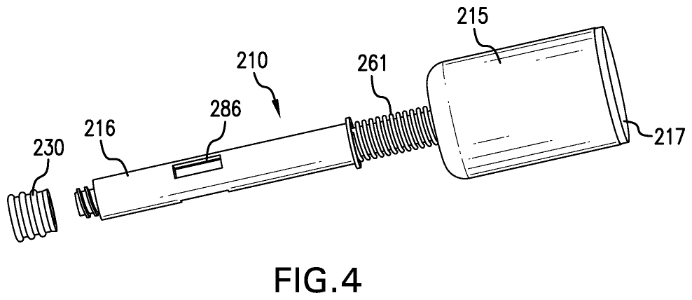

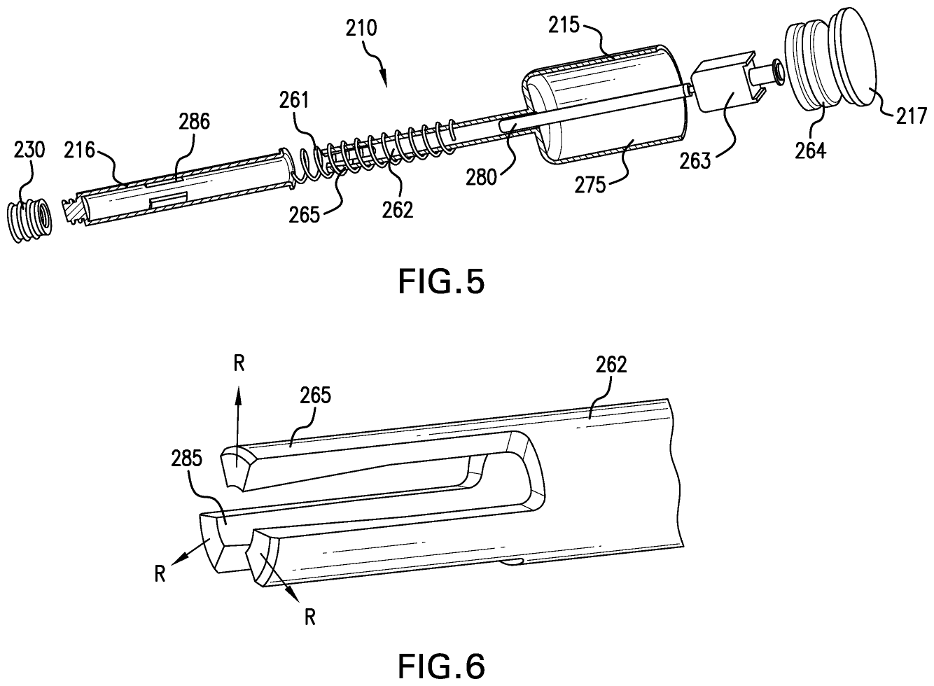

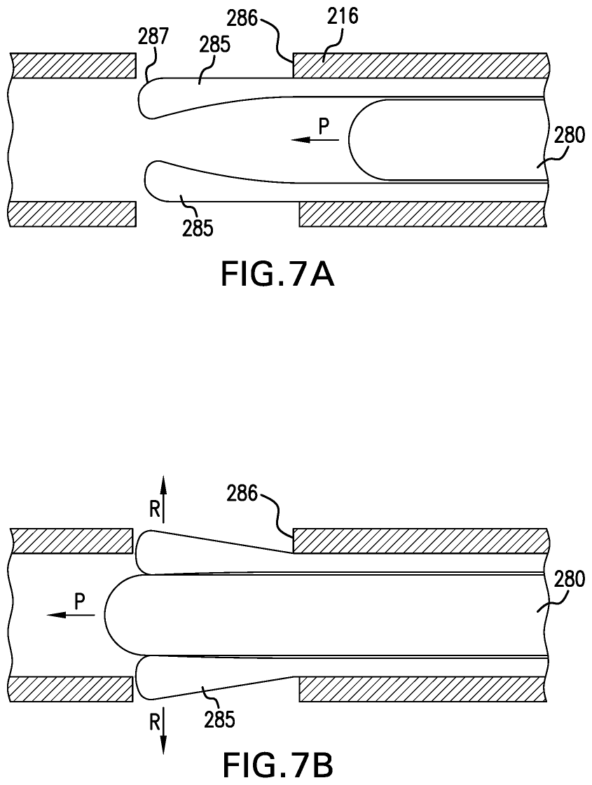

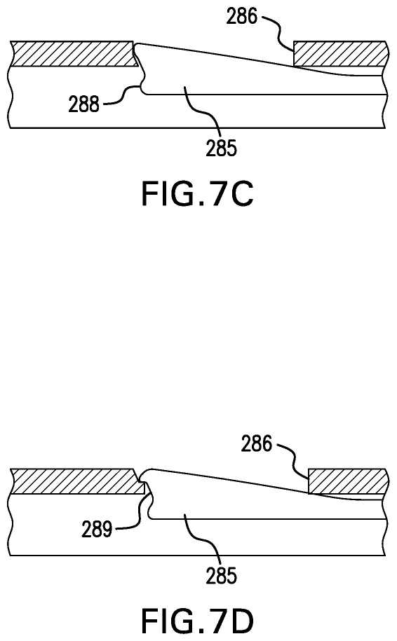

FIGS. 4-7B illustrate another embodiment of a plunger rod assembly 210. Here, the illustrated plunger rod assembly 210 includes pluralities of like-numbered elements that are substantially similar to those described above with reference to FIGS. 2-3, except that the elements are preceded by a “2” instead of a “1”. For example, plunger flange 217 corresponds to plunger flange 117 of FIG. 3 and may be constructed in a similar fashion. Flange 217 may be disposed over batteries 264 electrically in communication with the solenoid to power it and the device. In this example, latch 262 including fingers 265 may be unitarily formed with, attached to, and/or extend from the distal end of piston 215, and solenoid 263 may have a pin 280 that extends from the core of the solenoid for almost the full length of latch fingers 265. In this embodiment, the latch fingers 265 extend through spring 261 and may move freely along the length of housing 216 when solenoid 263 is not activated. Conversely, when solenoid 263 is activated, pin 280 may translate distally toward stopper 230 so that its distal end engages the inner surface 285 of the distal end of latch fingers 265. The inner surfaces 285 of latch fingers 265 may be shaped so that pin 280 forces them radially outward in the direction of arrow “R” when the pin moves distally therethrough. This translating movement of pin 280, shown with arrow “P” in FIG. 7A, causes the distal ends of latch fingers 265 to positively engage the mating features 286 in housing 216 (See, FIGS. 7A-B), whereby housing 216 and the piston become rigidly coupled with respect to travel in the distal direction. In some embodiments, the distal faces of latch fingers 285 comprise rounded or sloping features 287 that allow latch fingers to smoothly travel past mating features 286 when the solenoid is not activated (i.e., when pin 280 is not at least partially disposed inside or adjacent thereto).

In one embodiment, the distal faces of latch fingers 285 may also include features that cause them to positively engage the mating features in housing 216, whereby the housing 216 and piston 215 are rigidly mated when the solenoid is activated. In alternate embodiments, the distal faces of latch fingers 285 may include undercuts 288 (FIG. 7C), steps 289 (FIG. 7D) or similar features to create a positive lock or snap fit between the fingers and mating features 286 of housing 216 to maintain positive engagement between the housing and the piston.

In use, devices according to this disclosure may mitigate a potential misuse of dose splitting, which is dispensing and collecting at least part of the contents of the syringe instead of injecting it into a patient. The capacitive sensor may identify the presence of the needle in tissue or contact with the tissue. When the capacitive sensor is not activated the latch fingers are not engaged, and the piston can be axially displaced without the housing and stopper being displaced. When the capacitive sensor is activated, a signal is sent to the solenoid to actuate the latch fingers. With the latch fingers engaged, the housing and piston are locked together and axial translation of the piston results in distal motion of the housing and the stopper. In this manner, contents of the syringe may be ejected out of the needle. Such displacement of the stopper may only be possible when the capacitive sensor detects that the needle is embedded in tissue to prevent the contents of the pre-filled syringe from being transferred into another container for unintended use.

An additional benefit of these configurations is that they prevent accidental plunger movement before the injection of the drug into the patient. For example, during shipping or handling of the pre-filled syringe, accidental movement of the piston may occur from handling the syringe. This invention ensures that no dose can be lost or improperly dispensed before the syringe needle is inserted into a patient's tissue.

It is to be understood that the embodiments described herein are merely illustrative of the principles and applications of the present disclosure. For example, the number, positioning and arrangement of electrodes of the capacitance sensor, the piston, the solenoid, the latch, the latch fingers may be varied. Moreover, certain components are optional, and the disclosure contemplates various configurations and combinations of the elements disclosed herein. It is therefore to be understood that numerous modifications may be made to the illustrative embodiments and that other arrangements may be devised without departing from the spirit and scope of the present disclosure as defined by the appended claims.

It will be appreciated that the various dependent claims and the features set forth therein can be combined in different ways than presented in the initial claims. It will also be appreciated that the features described in connection with individual embodiments may be shared with others of the described embodiments.

1. An injecting device comprising:
a reservoir for containing a medicament;
a needle in communication with the reservoir and configured to deliver the medicament to a patient's body;
a capacitance sensor disposed adjacent to the needle; and
a plunger rod assembly comprising a housing and a piston, the housing and piston being configured to transition between a locked condition in which the piston and the housing are coupled to move together, and an unlocked condition in which the piston is capable of translating relative to the housing.

2. The injection device of claim 1, wherein the piston comprises a first portion and a second portion, the first portion having a first diameter and the second portion forming an enlarged cylindrical casing with a second diameter, the second diameter being greater than the first diameter.

3. The injection device of claim 2, further comprising a solenoid in communication with the capacitance sensor.

4. The injection device of claim 3, further comprising a latch operatively coupled to the solenoid.

5. The injection device of claim 4, wherein the latch comprises at least one finger.

6. The injection device of claim 4, wherein the latch comprises a plurality of fingers.

7. The injection device of claim 4, wherein the latch is disposed within the piston and translatable relative thereto.

8. The injection device of claim 4, further comprising a spring disposed within the housing, the spring being compressible by the piston.

9. The injection device of claim 4, wherein the piston comprises at least one opening for receiving a portion of the latch.

10. The injection device of claim 4, wherein the latch is coupled to the solenoid via a pin that travels through the latch.

11. The injection device of claim 4, wherein the latch and the pin are unitarily formed.

12. An injecting device comprising:
a reservoir for containing a medicament;
a needle in communication with the reservoir and configured to deliver the medicament to a patient's body;
a capacitance sensor disposed adjacent to the needle; and
a plunger rod assembly comprising a housing, a piston and a solenoid configured to transition the plunger rod assembly between a locked condition in which the piston and the housing move together, and an unlocked condition in which the piston is capable of translating relative to the housing.

13. The injection device of claim 12, further comprising a latch coupled to the solenoid.

14. The injection device of claim 13, wherein the latch comprises at least one finger.

15. The injection device of claim 13, wherein the latch comprises two, three, four or five fingers.

16. A method of administering a medicament, comprising:
providing an injecting device comprising: (i) a reservoir for containing a medicament, (ii) a needle in communication with the reservoir and configured to deliver the medicament to a patient's body, (iii) a capacitance sensor disposed adjacent to the needle, and (iv) a plunger rod assembly comprising a housing and a piston, the housing and piston being configured to transition between a locked condition in which the piston and the housing are coupled to move together, and an unlocked condition in which the piston is capable of translating relative to the housing;
measuring a first baseline capacitance via the capacitance sensor when the needle is not in physical contact with a patient's body; and
measuring a second capacitance via the capacitance sensor when the needle is in physical contact with the patient's body; and
determining if the needle was inserted in the patient's body based on a difference between the first baseline capacitance and the second capacitance.

17. The method of claim 16, wherein the plunger rod assembly of (iv) further comprises (v) a solenoid in communication with the capacitance sensor of (iii); and (vi) a latch operatively coupled to the solenoid.

18. The method of claim 16, wherein measuring a second capacitance comprises continuously measuring the second capacitance and comparing it to the first baseline capacitance until the difference between the first capacitance and the second capacitance is above a predetermined threshold.

19. The method of claim 16, further comprising transitioning the plunger rod assembly to the locked condition when the needle is in contact with the patient's body.

20. The method of claim 16, further comprising transitioning the plunger rod assembly to the unlocked condition when the needle is not in contact with the patient's body.
